SEQUENCE LISTING (15 sequences)

SEQ ID NO: 1 - DNA, 1455 bp, Bacillus sp.
  CDS (1)..(1455): SP690
catcataatggaacaaatggtactatgatgcaatatttcgaatggtatttgccaaatgacgggaatcattggaacaggttgagggatgacgcagctaacttaaagagtaaagggataacagctgtatggatcccacctgcatggaaggggacttcccagaatgatgtaggttatggagcctatgatttatatgatcttggagagtttaaccagaaggggacggttcgtacaaaatatggaacacgcaaccagctacaggctgcggtgacctctttaaaaaataacggcattcaggtatatggtgatgtcgtcatgaatcataaaggtggagcagatggtacggaaattgtaaatgcggtagaagtgaatcggagcaaccgaaaccaggaaacctcaggagagtatgcaatagaagcgtggacaaagtttgattttcctggaagaggaaataaccattccagctttaagtggcgctggtatcattttgatgggacagattgggatcagtcacgccagcttcaaaacaaaatatataaattcaggggaacaggcaaggcctgggactgggaagtcgatacagagaatggcaactatgactatcttatgtatgcagacgtggatatggatcacccagaagtaatacatgaacttagaaactggggagtgtggtatacgaatacactgaaccttgatggatttagaatagatgcagtgaaacatataaaatatagctttacgagagattggcttacacatgtgcgtaacaccacaggtaaaccaatgtttgcagtggctgagttttggaaaaatgaccttggtgcaattgaaaactatttgaataaaacaagttggaatcactcggtgtttgatgttcctctccactataatttgtacaatgcatctaatagcggtggttattatgatatgagaaatattttaaatggttctgtggtgcaaaaacatccaacacatgccgttacttttgttgataaccatgattctcagcccggggaagcattggaatcctttgttcaacaatggtttaaaccacttgcatatgcattggttctgacaagggaacaaggttatccttccgtattttatggggattactacggtatcccaacccatggtgttccggctatgaaatctaaaatagaccctcttctgcaggcacgtcaaacttttgcctatggtacgcagcatgattactttgatcatcatgatattatcggttggacaagagagggaaatagctcccatccaaattcaggccttgccaccattatgtcagatggtccaggtggtaacaaatggatgtatgtggggaaaaataaagcgggacaagtttggagagatattaccggaaataggacaggcaccgtcacaattaatgcagacggatggggtaatttctctgttaatggagggtccgtttcggtttgggtgaagcaa

SEQ ID NO: 2 - protein, 485 aa, Bacillus sp.
HHNGTNGTMMQYFEWYLPNDGNHWNRLRDDAANLKSKGITAVWIPPAWKGTSQNDVGYGAYDLYDLGEFNQKGTVRTKYGTRNQLQAAVTSLKNNGIQVYGDVVMNHKGGADGTEIVNAVEVNRSNRNQETSGEYAIEAWTKFDFPGRGNNHSSFKWRWYHFDGTDWDQSRQLQNKIYKFRGTGKAWDWEVDTENGNYDYLMYADVDMDHPEVIHELRNWGVWYTNTLNLDGFRIDAVKHIKYSFTRDWLTHVRNTTGKPMFAVAEFWKNDLGAIENYLNKTSWNHSVFDVPLHYNLYNASNSGGYYDMRNILNGSVVQKHPTHAVTFVDNHDSQPGEALESFVQQWFKPLAYALVLTREQGYPSVFYGDYYGIPTHGVPAMKSKIDPLLQARQTFAYGTQHDYFDHHDIIGWTREGNSSHPNSGLATIMSDGPGGNKWMYVGKNKAGQVWRDITGNRTGTVTINADGWGNFSVNGGSVSVWVKQ

SEQ ID NO: 3 - DNA, 1455 bp, Bacillus sp.
  CDS (1)..(1455): SP722
catcataatgggacaaatgggacgatgatgcaatactttgaatggcacttgcctaatgatgggaatcactggaatagattaagagatgatgctagtaatctaagaaatagaggtataaccgctatttggattccgcctgcctggaaagggacttcgcaaaatgatgtggggtatggagcctatgatctttatgatttaggggaatttaatcaaaaggggacggttcgtactaagtatgggacacgtagtcaattggagtctgccatccatgctttaaagaataatggcgttcaagtttatggggatgtagtgatgaaccataaaggaggagctgatgctacagaaaacgttcttgctgtcgaggtgaatccaaataaccggaatcaagaaatatctggggactacacaattgaggcttggactaagtttgattttccagggaggggtaatacatactcagactttaaatggcgttggtatcatttcgatggtgtagattgggatcaatcacgacaattccaaaatcgtatctacaaattccgaggtgatggtaaggcatgggattgggaagtagattcggaaaatggaaattatgattatttaatgtatgcagatgtagatatggatcatccggaggtagtaaatgagcttagaagatggggagaatggtatacaaatacattaaatcttgatggatttaggatcgatgcggtgaagcatattaaatatagctttacacgtgattggttgacccatgtaagaaacgcaacgggaaaagaaatgtttgctgttgctgaattttggaaaaatgatttaggtgccttggagaactatttaaataaaacaaactggaatcattctgtctttgatgtcccccttcattataatctttataacgcgtcaaatagtggaggcaactatgacatggcaaaacttcttaatggaacggttgttcaaaagcatccaatgcatgccgtaacttttgtggataatcacgattctcaacctggggaatcattagaatcatttgtacaagaatggtttaagccacttgcttatgcgcttattttaacaagagaacaaggctatccctctgtcttctatggtgactactatggaattccaacacatagtgtcccagcaatgaaagccaagattgatccaatcttagaggcgcgtcaaaattttgcatatggaacacaacatgattattttgaccatcataatataatcggatggacacgtgaaggaaataccacgcatcccaattcaggacttgcgactatcatgtcggatgggccagggggagagaaatggatgtacgtagggcaaaataaagcaggtcaagtttggcatgacataactggaaataaaccaggaacagttacgatcaatgcagatggatgggctaatttttcagtaaatggaggatctgtttccatttgggtgaaacga

SEQ ID NO: 4 - protein, 485 aa, Bacillus sp.
HHNGTNGTMMQYFEWHLPNDGNHWNRLRDDASNLRNRGITAIWIPPAWKGTSQNDVGYGAYDLYDLGEFNQKGTVRTKYGTRSQLESAIHALKNNGVQVYGDVVMNHKGGADATENVLAVEVNPNNRNQEISGDYTIEAWTKFDFPGRGNTYSDFKWRWYHFDGVDWDQSRQFQNRIYKFRGDGKAWDWEVDSENGNYDYLMYADVDMDHPEVVNELRRWGEWYTNTLNLDGFRIDAVKHIKYSFTRDWLTHVRNATGKEMFAVAEFWKNDLGALENYLNKTNWNHSVFDVPLHYNLYNASNSGGNYDMAKLLNGTVVQKHPMHAVTFVDNHDSQPGESLESFVQEWFKPLAYALILTREQGYPSVFYGDYYGIPTHSVPAMKAKIDPILEARQNFAYGTQHDYFDHHNIIGWTREGNTTHPNSGLATIMSDGPGGEKWMYVGQNKAGQVWHDITGNKPGTVTINADGWANFSVNGGSVSIWVKR

SEQ ID NO: 5 - DNA, 1548 bp, Bacillus stearothermophilus
  CDS (1)..(1548): BSG
gccgcaccgtttaacggcaccatgatgcagtattttgaatggtacttgccggatgatggcacgttatggaccaaagtggccaatgaagccaacaacttatccagccttggcatcaccgctctttggctgccgcccgcttacaaaggaacaagccgcagcgacgtagggtacggagtatacgacttgtatgacctcggcgaattcaatcaaaaagggaccgtccgcacaaaatacggaacaaaagctcaatatcttcaagccattcaagccgcccacgccgctggaatgcaagtgtacgccgatgtcgtgttcgaccataaaggcggcgctgacggcacggaatgggtggacgccgtcgaagtcaatccgtccgaccgcaaccaagaaatctcgggcacctatcaaatccaagcatggacgaaatttgattttcccgggcggggcaacacctactccagctttaagtggcgctggtaccattttgacggcgttgattgggacgaaagccgaaaattgagccgcatttacaaattccgcggcatcggcaaagcgtgggattgggaagtagacacggaaaacggaaactatgactacttaatgtatgccgaccttgatatggatcatcccgaagtcgtgaccgagctgaaaaactgggggaaatggtatgtcaacacaacgaacattgatgggttccggcttgatgccgtcaagcatattaagttcagtttttttcctgattggttgtcgtatgtgcgttctcagactggcaagccgctatttaccgtcggggaatattggagctatgacatcaacaagttgcacaattacattacgaaaacagacggaacgatgtctttgtttgatgccccgttacacaacaaattttataccgcttccaaatcagggggcgcatttgatatgcgcacgttaatgaccaatactctcatgaaagatcaaccgacattggccgtcaccttcgttgataatcatgacaccgaacccggccaagcgctgcagtcatgggtcgacccatggttcaaaccgttggcttacgcctttattctaactcggcaggaaggatacccgtgcgtcttttatggtgactattatggcattccacaatataacattccttcgctgaaaagcaaaatcgatccgctcctcatcgcgcgcagggattatgcttacggaacgcaacatgattatcttgatcactccgacatcatcgggtggacaagggaagggggcactgaaaaaccaggatccggactggccgcactgatcaccgatgggccgggaggaagcaaatggatgtacgttggcaaacaacacgctggaaaagtgttctatgaccttaccggcaaccggagtgacaccgtcaccatcaacagtgatggatggggggaattcaaagtcaatggcggttcggtttcggtttgggttcctagaaaaacgaccgtttctaccatcgctcggccgatcacaacccgaccgtggactggtgaattcgtccgttggaccgaaccacggttggtggcatggccttga

SEQ ID NO: 6 - protein, 515 aa, Bacillus stearothermophilus
AAPFNGTMMQYFEWYLPDDGTLWTKVANEANNLSSLGITALWLPPAYKGTSRSDVGYGVYDLYDLGEFNQKGTVRTKYGTKAQYLQAIQAAHAAGMQVYADVVFDHKGGADGTEWVDAVEVNPSDRNQEISGTYQIQAWTKFDFPGRGNTYSSFKWRWYHFDGVDWDESRKLSRIYKFRGIGKAWDWEVDTENGNYDYLMYADLDMDHPEVVTELKNWGKWYVNTTNIDGFRLDAVKHIKFSFFPDWLSYVRSQTGKPLFTVGEYWSYDINKLHNYITKTDGTMSLFDAPLHNKFYTASKSGGAFDMRTLMTNTLMKDQPTLAVTFVDNHDTEPGQALQSWVDPWFKPLAYAFILTRQEGYPCVFYGDYYGIPQYNIPSLKSKIDPLLIARRDYAYGTQHDYLDHSDIIGWTREGGTEKPGSGLAALITDGPGGSKWMYVGKQHAGKVFYDLTGNRSDTVTINSDGWGEFKVNGGSVSVWVPRKTTVSTIARPITTRPWTGEFVRWTEPRLVAWP

SEQ ID NO: 7 - DNA, 1920 bp, Bacillus licheniformis
  CDS (421)..(1872): TERMAMYL
cggaagattggaagtacaaaaataagcaaaagattgtcaatcatgtcatgagccatgcgggagacggaaaaatcgtcttaatgcacgatatttatgcaacgttcgcagatgctgctgaagagattattaaaaagctgaaagcaaaaggctatcaattggtaactgtatctcagcttgaagaagtgaagaagcagagaggctattgaataaatgagtagaagcgccatatcggcgcttttcttttggaagaaaatatagggaaaatggtacttgttaaaaattcggaatatttatacaacatcatatgtttcacattgaaaggggaggagaatcatgaaacaacaaaaacggctttacgcccgattgctgacgctgttatttgcgctcatcttcttgctgcctcattctgcagcagcggcggcaaatcttaatgggacgctgatgcagtattttgaatggtacatgcccaatgacggccaacattggaggcgtttgcaaaacgactcggcatatttggctgaacacggtattactgccgtctggattcccccggcatataagggaacgagccaagcggatgtgggctacggtgcttacgacctttatgatttaggggagtttcatcaaaaagggacggttcggacaaagtacggcacaaaaggagagctgcaatctgcgatcaaaagtcttcattcccgcgacattaacgtttacggggatgtggtcatcaaccacaaaggcggcgctgatgcgaccgaagatgtaaccgcggttgaagtcgatcccgctgaccgcaaccgcgtaatttcaggagaacacctaattaaagcctggacacattttcattttccggggcgcggcagcacatacagcgattttaaatggcattggtaccattttgacggaaccgattgggacgagtcccgaaagctgaaccgcatctataagtttcaaggaaaggcttgggattgggaagtttccaatgaaaacggcaactatgattatttgatgtatgccgacatcgattatgaccatcctgatgtcgcagcagaaattaagagatggggcacttggtatgccaatgaactgcaattggacggtttccgtcttgatgctgtcaaacacattaaattttcttttttgcgggattgggttaatcatgtcagggaaaaaacggggaaggaaatgtttacggtagctgaatattggcagaatgacttgggcgcgctggaaaactatttgaacaaaacaaattttaatcattcagtgtttgacgtgccgcttcattatcagttccatgctgcatcgacacagggaggcggctatgatatgaggaaattgctgaacggtacggtcgtttccaagcatccgttgaaatcggttacatttgtcgataaccatgatacacagccggggcaatcgcttgagtcgactgtccaaacatggtttaagccgcttgcttacgcttttattctcacaagggaatctggataccctcaggttttctacggggatatgtacgggacgaaaggagactcccagcgcgaaattcctgccttgaaacacaaaattgaaccgatcttaaaagcgagaaaacagtatgcgtacggagcacagcatgattatttcgaccaccatgacattgtcggctggacaagggaaggcgacagctcggttgcaaattcaggtttggcggcattaataacagacggacccggtggggcaaagcgaatgtatgtcggccggcaaaacgccggtgagacatggcatgacattaccggaaaccgttcggagccggttgtcatcaattcggaaggctggggagagtttcacgtaaacggcgggtcggtttcaatttatgttcaaagatagaagagcagagaggacggatttcctgaaggaaatccgtttttttatttt

SEQ ID NO: 8 - protein, 483 aa, Bacillus licheniformis [sequence omitted]

SEQ ID NO: 9 - DNA, 2084 bp, Bacillus amyloliquefaciens
  CDS (343)..(1794): BAN
gccccgcacatacgaaaagactggctgaaaacattgagcctttgatgactgatgatttggctgaagaagtggatcgattgtttgagaaaagaagaagaccataaaaataccttgtctgtcatcagacagggtattttttatgctgtccagactgtccgctgtgtaaaaataaggaataaaggggggttgttattattttactgatatgtaaaatataatttgtataagaaaatgagagggagaggaaacatgattcaaaaacgaaagcggacagtttcgttcagacttgtgcttatgtgcacgctgttatttgtcagtttgccgattacaaaaacatcagccgtaaatggcacgctgatgcagtattttgaatggtatacgccgaacgacggccagcattggaaacgattgcagaatgatgcggaacatttatcggatatcggaatcactgccgtctggattcctcccgcatacaaaggattgagccaatccgataacggatacggaccttatgatttgtatgatttaggagaattccagcaaaaagggacggtcagaacgaaatacggcacaaaatcagagcttcaagatgcgatcggctcactgcattcccggaacgtccaagtatacggagatgtggttttgaatcataaggctggtgctgatgcaacagaagatgtaactgccgtcgaagtcaatccggccaatagaaatcaggaaacttcggaggaatatcaaatcaaagcgtggacggattttcgttttccgggccgtggaaacacgtacagtgattttaaatggcattggtatcatttcgacggagcggactgggatgaatcccggaagatcagccgcatctttaagtttcgtggggaaggaaaagcgtgggattgggaagtatcaagtgaaaacggcaactatgactatttaatgtatgctgatgttgactacgaccaccctgatgtcgtggcagagacaaaaaaatggggtatctggtatgcgaatgaactgtcattagacggcttccgtattgatgccgccaaacatattaaattttcatttctgcgtgattgggttcaggcggtcagacaggcgacgggaaaagaaatgtttacggttgcggagtattggcagaataatgccgggaaactcgaaaactacttgaataaaacaagctttaatcaatccgtgtttgatgttccgcttcatttcaatttacaggcggcttcctcacaaggaggcggatatgatatgaggcgtttgctggacggtaccgttgtgtccaggcatccggaaaaggcggttacatttgttgaaaatcatgacacacagccgggacagtcattggaatcgacagtccaaacttggtttaaaccgcttgcatacgcctttattttgacaagagaatccggttatcctcaggtgttctatggggatatgtacgggacaaaagggacatcgccaaaggaaattccctcactgaaagataatatagagccgattttaaaagcgcgtaaggagtacgcatacgggccccagcacgattatattgaccacccggatgtgatcggatggacgagggaaggtgacagctccgccgccaaatcaggtttggccgctttaatcacggacggacccggcggatcaaagcggatgtatgccggcctgaaaaatgccggcgagacatggtatgacataacgggcaaccgttcagatactgtaaaaatcggatctgacggctggggagagtttcatgtaaacgatgggtccgtctccatttatgttcagaaataaggtaataaaaaaacacctccaagctgagtgcgggtatcagcttggaggtgcgtttattttttcagccgtatgacaaggtcggcatcaggtgtgacaaatacggtatgctggctgtcataggtgacaaatccgggttttgcgccgtttggctttttcacatgtctgatttttgtataatcaacaggcacggagccggaatctttcgccttggaaaaataagcggcgatcgtagctgcttccaatatggattgttcatcgggatcgctgcttttaatcacaacgtgggatcc

SEQ ID NO: 10 - protein, 483 aa, Bacillus amyloliquefaciens
VNGTLMQYFEWYTPNDGQHWKRLQNDAEHLSDIGITAVWIPPAYKGLSQSDNGYGPYDLYDLGEFQQKGTVRTKYGTKSELQDAIGSLHSRNVQVYGDVVLNHKAGADATEDVTAVEVNPANRNQETSEEYQIKAWTDFRFPGRGNTYSDFKWHWYHFDGADWDESRKISRIFKFRGEGKAWDWEVSSENGNYDYLMYADVDYDHPDVVAETKKWGIWYANELSLDGFRIDAAKHIKFSFLRDWVQAVRQATGKEMFTVAEYWQNNAGKLENYLNKTSFNQSVFDVPLHFNLQAASSQGGGYDMRRLLDGTVVSRHPEKAVTFVENHDTQPGQSLESTVQTWFKPLAYAFILTRESGYPQVFYGDMYGTKGTSPKEIPSLKDNIEPILKARKEYAYGPQHDYIDHPDVIGWTREGDSSAAKSGLAALITDGPGGSKRMYAGLKNAGETWYDITGNRSDTVKIGSDGWGEFHVNDGSVSIYVQK

SEQ ID NO: 11 - DNA, 1458 bp, Bacillus sp.
  CDS (1)..(1458): AA560
caccataatggtacgaacggcacaatgatgcagtactttgaatggtatctaccaaatgacggaaaccattggaatagattaaggtctgatgcaagtaacctaaaagataaagggatctcagcggtttggattcctcctgcatggaagggtgcctctcaaaatgatgtggggtatggtgcttatgatctgtatgatttaggagaattcaatcaaaaaggaaccattcgtacaaaatatggaacgcgcaatcagttacaagctgcagttaacgccttgaaaagtaatggaattcaagtgtatggcgatgttgtaatgaatcataaagggggagcagacgctaccgaaatggttagggcagttgaagtaaacccgaataatagaaatcaagaagtgtccggtgaatatacaattgaggcttggacaaagtttgactttccaggacgaggtaatactcattcaaacttcaaatggagatggtatcactttgatggagtagattgggatcagtcacgtaagctgaacaatcgaatttataaatttagaggtgatggaaaagggtgggattgggaagtcgatacagaaaacggtaactatgattacctaatgtatgcagatattgacatggatcacccagaggtagtgaatgagctaagaaattggggtgtttggtatacgaatacattaggccttgatggttttagaatagatgcagtaaaacatataaaatacagctttactcgtgattggattaatcatgttagaagtgcaactggcaaaaatatgtttgcggttgcggaattttggaaaaatgatttaggtgctattgaaaactatttaaacaaaacaaactggaaccattcagtctttgatgttccgctgcactataacctctataatgcttcaaaaagcggagggaattatgatatgaggcaaatatttaatggtacagtcgtgcaaagacatccaatgcatgctgttacatttgttgataatcatgattcgcaacctgaagaagctttagagtcttttgttgaagaatggttcaaaccattagcgtatgctttgacattaacacgtgaacaaggctacccttctgtattttatggagattattatggcattccaacgcatggtgtaccagcgatgaaatcgaaaattgacccgattctagaagcgcgtcaaaagtatgcatatggaagacaaaatgactacttagaccatcataatatcatcggttggacacgtgaagggaatacagcacaccccaactccggtttagctactatcatgtccgatggggcaggaggaaataagtggatgtttgttgggcgtaataaagctggtcaagtttggaccgatatcactggaaatcgtgcaggtactgttacgattaatgctgatggatggggtaatttttctgtaaatggaggatcagtttctatttgggtaaacaaataa

SEQ ID NO: 12 - protein, 485 aa, Bacillus sp.
HHNGTNGTMMQYFEWYLPNDGNHWNRLRSDASNLKDKGISAVWIPPAWKGASQNDVGYGAYDLYDLGEFNQKGTIRTKYGTRNQLQAAVNALKSNGIQVYGDVVMNHKGGADATEMVRAVEVNPNNRNQEVSGEYTIEAWTKFDFPGRGNTHSNFKWRWYHFDGVDWDQSRKLNNRIYKFRGDGKGWDWEVDTENGNYDYLMYADIDMDHPEVVNELRNWGVWYTNTLGLDGFRIDAVKHIKYSFTRDWINHVRSATGKNMFAVAEFWKNDLGAIENYLNKTNWNHSVFDVPLHYNLYNASKSGGNYDMRQIFNGTVVQRHPMHAVTFVDNHDSQPEEALESFVEEWFKPLAYALTLTREQGYPSVFYGDYYGIPTHGVPAMKSKIDPILEARQKYAYGRQNDYLDHHNIIGWTREGNTAHPNSGLATIMSDGAGGNKWMFVGRNKAGQVWTDITGNRAGTVTINADGWGNFSVNGGSVSIWVNK

SEQ ID NO: 13 - protein, 485 aa, Bacillus 707
HHNGTNGTMMQYFEWYLPNDGNHWNRLNSDASNLKSKGITAVWIPPAWKGASQNDVGYGAYDLYDLGEFNQKGTVRTKYGTRSQLQAAVTSLKNNGIQVYGDVVMNHKGGADATEMVRAVEVNPNNRNQEVTGEYTIEAWTRFDFPGRGNTHSSFKWRWYHFDGVDWDQSRRLNNRIYKFRGHGKAWDWEVDTENGNYDYLMYADIDMDHPEVVNELRNWGVWYTNTLGLDGFRIDAVKHIKYSFTRDWINHVRSATGKNMFAVAEFWKNDLGAIENYLQKTNWNHSVFDVPLHYNLYNASKSGGNYDMRNIFNGTVVQRHPSHAVTFVDNHDSQPEEALESFVEEWFKPLAYALTLTREQGYPSVFYGDYYGIPTHGVPAMRSKIDPILEARQKYAYGKQNDYLDHHNIIGWTREGNTAHPNSGLATIMSDGAGGSKWMFVGRNKAGQVWSDITGNRTGTVTINADGWGNFSVNGGSVSIWVNK

SEQ ID NO: 14 - DNA, 20 bp, Artificial Sequence
  misc_feature (1)..(20): Forward Primer
gacctgcagtcaggcaacta

SEQ ID NO: 15 - DNA, 20 bp, Artificial Sequence
  misc_feature (1)..(20): Reverse Primer
tagagtcgacctgcaggcat

(19) United States
(12) Patent Application Publication
(10) Pub. No.: US 2002/0068352 A1
(43) Pub. Date: Jun. 6, 2002

(54) Alpha-amylase variants with altered 1, 6-activity

(75) Inventors: Allan Svendsen, Horsholm (DK); Christel Thea Jorgensen, Kobenhavn O (DK); Bjarne Ronfeldt Nielsen, Virum (DK)

Correspondence Address: NOVOZYMES NORTH AMERICA, INC., C/O NOVO NORDISK OF NORTH AMERICA, INC., 405 LEXINGTON AVENUE, SUITE 6400, NEW YORK, NY, 10174, US

(73) Assignee: Novozymes A/S, Bagsvaerd (DK)

(21) Appl. No.: 09/854,346

(22) Filed: May 11, 2001

Related U.S. Application Data
(60) Provisional application No. 60/205,229, filed on May 17, 2000.

(30) Foreign Application Priority Data
May 12, 2000 (DK) PA 2000 00779

(51) Int. Cl. C12N 9/28

(52) U.S. Cl. 435/202; 435/183; 435/195; 435/69.1; 510/392; 510/393

(57) ABSTRACT

The present invention relates to variants of parent Termamyl-like alpha-amylases, which variant has alpha-amylase activity and exhibits an alteration in the alpha-1,6-D-glucosidic branch linkage cleavage activity of amylopectin and limit dextrins.

### CROSS-REFERENCE TO RELATED APPLICATIONS

This application claims, under 35 U.S.C. 119, priority of Danish application Ser. No. PA 2000 00779 filed May 12, 2000, and the benefit of U.S. provisional application No. 60/205,229, filed May 17, 2000, the contents of which are fully incorporated herein by reference.

### FIELD OF THE INVENTION

The present invention relates to variants (mutants) of parent Termamyl-like alpha-amylases, which variant exhibits an alteration in alpha-1,6-D-glucosidic branch linkage cleavage activity. The invention also relates a DNA construct comprising a DNA sequence encoding the alpha-amylase variant of the invention, an expression vector for recombinant production and a host cell for recombinant production. The invention also relates to various compositions comprising a variant of the invention, especially for use in the liquefaction step of, e.g., a strach convention or ethanol process, and finally to the use of such variants or compositions of the invention for various uses.

### BACKGROUND OF THE INVENTION

Alpha-Amylases (alpha-1,4-glucan-4-glucanohydrolases, E.C. 3.2.1.1) constitute a group of enzymes, which catalyze hydrolysis of starch and other linear and branched 1,4-glucosidic oligo-and polysaccharides.

Protein engineering is increasingly used to alter the properties of enzymes—such as alpha-amylase-to obtain an enzyme with properties tailored for specific applications.

### BRIEF DISCLOSURE OF THE INVENTION

The object of the present invention is to provide Termamyl-like amylases which variants in comparison to the corresponding parent alpha-amylase, i.e., un-mutated alpha-amylase, has alpha-amylase activity and exhibits an alteration in alpha-1,6-D-glucosidic branch linkage cleavage activity of amylopectin and alpha- and beta-limit dextrins.

Alpha-amylases with altered activity towards alpha-1,6-D-glucosidic branch linkages of amylopectin and alpha- and beta-limit dextrins are desired, because such enzyme variants may, if the 1,6-activity is increased, increase the glucose yield in the liquefaction process in connection with, e.g., high fructose corn syrup (HFCS) production, because less panose will be formed. Further, in detergents the degradation of starch into smaller sugar units (less limit dextrins or smaller limit dextrins) facilitates the washing out of the sugar molecules and thus improve the wash performance. If a higher limit dextrins level is desired reduced 1,6-activity is advantageous.

### Nomenclature

In the present description and claims, the conventional one-letter and three-letter codes for amino acid residues are used. For ease of reference, alpha-amylase variants of the invention are described by use of the following nomenclature:

Original amino acid(s): position(s): substituted amino acid(s)

According to this nomenclature, for instance the substitution of alanine for asparagine in position 30 is shown as:

Ala30Asn or A30N

a deletion of alanine in the same position is shown as:

Ala30* or A30*

and insertion of an additional amino acid residue, such as lysine, is shown as:

Ala30AlaLys or A30AK

A deletion of a consecutive stretch of amino acid residues, such as amino acid residues 30-33, is indicated as (30-33)* or Δ(A30-N33).

Where a specific alpha-amylase contains a “deletion” in comparison with other alpha-amylases and an insertion is made in such a position this is indicated as:

*36Asp or *36D for insertion of an aspartic acid in position 36. Multiple mutations are separated by plus signs, i.e.:

Ala30Asp+Glu34Ser or A30N+E34S representing mutations in positions 30 and 34 substituting alanine and glutamic acid for asparagine and serine, respectively.

When one or more alternative amino acid residues may be inserted in a given position it is indicated as

A30N,E or

A30N or A30E

Furthermore, when a position suitable for modification is identified herein without any specific modification being suggested, it is to be understood that any amino acid residue may be substituted for the amino acid residue present in the position. Thus, for instance, when a modification of an alanine in position 30 is mentioned, but not specified, it is to be understood that the alanine may be deleted or substituted for any other amino acid, i.e., any one of:

R,N,D,C,Q,E,G,H,I,L,K,M,F,P,S,T,W,Y,V.

Further, “A30X” means any one of the following substitutions: A30R, A30N, A30D, A30C, A30Q, A30E, A30G, A30H, A30I, A30L, A30K, A30M, A30F, A30P, A30S, A30T, A30W, A30Y, or A30 V; or in short: A30R,N,D,C,Q,E,G,H,I,L,K,M,F,P,S,T,W,Y,V.

In the first aspect the invention relates a variant of a parent Termamyl-like alpha-amylase, comprising an alteration at one or more of the following regions or positions selected from the group of:

Region: 186-193,

Region: 261-276,

Region: 283-293,

Region: 334-339,

Position: 234, wherein

(a) the alteration(s) are independently

(i) an insertion of an amino acid downstream of the amino acid which occupies the position,

(ii) a deletion of the amino acid which occupies the position, or

(iii) a substitution of the amino acid which occupies the position with a different amino acid,

(b) the variant has alpha-amylase activity and (c) each position corresponds to a position of the amino acid sequence of the parent Termamyl-like alpha-amylase having the B. licheniformis alpha-amylase amino acid sequence of shown in SEQ ID NO: 8.

The invention also relates to a number of specific variants; to a DNA construct comprising a DNA sequence encoding an alpha-amylase variant of the invention; a recombinant expression vector which carries a DNA construct of the invention; a cell which is transformed with a DNA construct of the invention or a vector of the invention; composition comprising alpha-amylase variant of the invention; the use of a variant of the invention for specified industrial applications.

### BRIEF DESCRIPTION OF THE DRAWINGS

FIG. 1 is an alignment of the amino acid sequences of five parent Termamyl-like alpha-amylases. The numbers on the extreme left designate the respective amino acid sequences as follows:

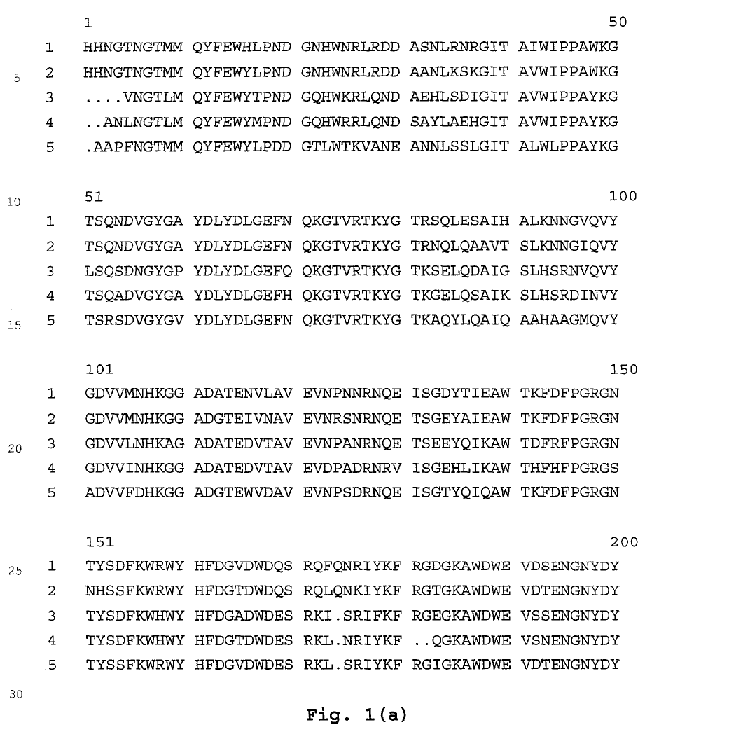

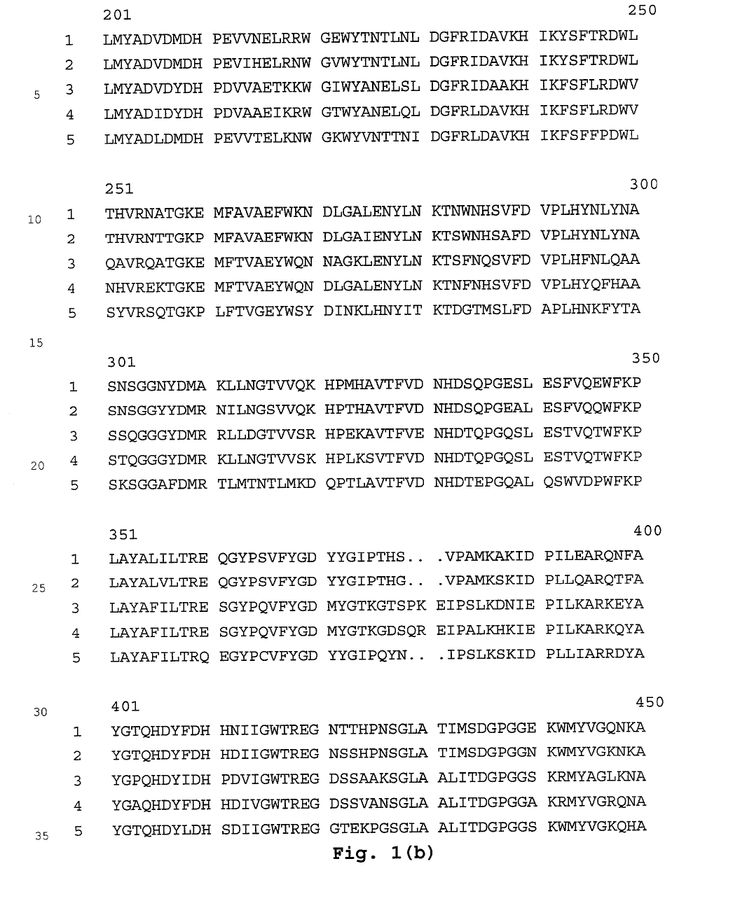

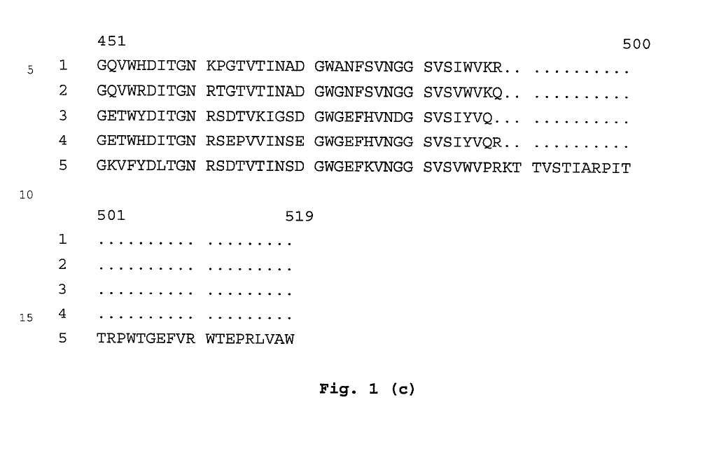

1: SEQ ID NO: 2

2: SEQ ID NO: 1

3: SEQ ID NO: 5

4: SEQ ID NO: 4

5: SEQ ID NO: 3.

### DETAILED DISCLOSURE OF THE INVENTION

The object of the present invention is to provide Termamyl-like amylases, which variants exhibits an alteration in the alpha-1,6-D-glucosidic branch linkage cleavage activity of especially amylopectin and alpha—and/or beta—limit dextrins.

### Altered Alpha-1,6-D-Glucosidic Branch Linkage Activity

Alpha-amylases, which catalyze hydrolysis of starch and other linear and branched 1,4-glucosidic oligo—and polysaccharides have in general limited activity on the alpha-1,6-D-glucosidic branch linkages of amylopectin and alpha- and beta-limit dextrins.

For instance, to increase the final dextrose yield in starch convention processes it is desirable to break the alpha-1,6-D-glucosidic branch linkages in amylopectin and alpha—and/or beta-limit dextrins.

If an increased amount of alpha—and/or beta-limit dextrins is desired it may be advantageous to decrease the alpha-1,6-D-glucosidic branch linkages activity.

The present inventors have found that the alpha-1,6-D-glucosidic branch linkages of amylopectin and/or alpha—and/or beta-limit dextrins can be altered by mutating within one or more of the below mentioned regions or positions in Termamyl-like alpha-amylases.

Thus, in the first aspect the invention relates a variant of a parent Termamyl-like alpha-amylase, comprising an alteration at one or more of the following regions or positions selected from the group of:

Region: 186-193,

Region: 261-276,

Region: 283-293,

Region: 334-339,

Position 234,

wherein

(a) the alteration(s) are independently

(i) an insertion of an amino acid downstream of the amino acid which occupies the position,

(ii) a deletion of the amino acid which occupies the position, or

(iii) a substitution of the amino acid which occupies the position with a different amino acid,

(b) the variant has alpha-amylase activity and (c) each position corresponds to a position of the amino acid sequence of the parent Termamyl-like alpha-amylase having the B. licheniformis alpha-amylase amino acid sequence of shown in SEQ ID NO: 8.

In an embodiment the region mutated is the Region: 186-193. Specific preferred positions contemplated include one or more of positions 186, 187, 188, 189, 190, 191, 192, 193. Specific mutations include one or more of: E189GASTV;

In another embodiment the region mutated is the Region: 261-276. Specific preferred positions contemplated include one or more of positions 261, 262, 263, 264, 265, 266, 266, 267, 268, 269, 270, 271, 272, 273, 274, 275, 276. Specific mutations include one or more of:

W263GASTV; Q264X; N265GASTV;

In another embodiment the region mutated is the Region: 283-293. Specific preferred positions contemplated include one or more of positions 283, 284, 285, 286, 287, 288, 289, 290, 291, 292, 293. Specific mutations include one or more of: V286FWY or smaller residues than V, e.g., GAS; Y290 smaller residues than Y, e.g., A,N,D,C,Q,E,G,H,I,L,K,M,F,P,S,T,V.

In another embodiment the region mutated is the Region: 334-339. Specific preferred positions contemplated include one or more of positions 334, 335, 336, 337, 338, 339. Specific mutations include one or more of: L335G,A,S,T,V;

Finally, position K234 is also contemplated according to the invention, preferably K234X, especially preferably K234N,Q.

### Detection of 1,6-Activity of Alpha-Amylases

In the presence of 1,4-activity, additional 1,6-activity can be measured by NMR spectroscopic methods. ¹H NMR spectroscopy allows a direct measure of the relative amounts of the anomeric protons of alpha(1→4)-linkages, alpha(1→6)-linkages and reducing ends, respectively. With a suitable substrate the hydrolysis of the individual bond types can be followed during reaction, e.g., hydrolysis of alpha(1→4)-linkages will give a reduction in the integral of the alpha(1→4)-signals and an equivalent increase in the integral of the signals coming from the reducing ends. In case of an enzyme, which have a supplementary 1,6-activity it is useful to look at the ratio R—(Iₐₗₚₕₐ(1→4)+I_{red. ends})/Iₐₗₚₕₐ(1→6), where I is the integral. If only alpha(1→4)-linkages are being hydrolysed R will remain constant throughout the reaction. If alpha(1→6)-linkages are also being cleaved R will increase.

Synthetic designed substrates and limit dextrins can be used as substrates. Termamyl® (B. licehniformis alpha-amylase shown in SEQ ID NO: 8) and Novamyl® (B. stearothermophilus C599 maltogenic amylase disclosed in EP patent no. 120,693 (Novo Industri A/S) limit dextrins (made from amylopectin), where the low molecular weight material (<1000 Da) has been removed by ultrafiltration may be used. The incubations can be followed directly in the NMR tube (D₂O) at 60° C. if enzyme and substrate are pretreated repeatedly with D₂O.

An NMR procedure for detection of 1,6 activity is described below in “Materials & Methods” section.

Limit dextrins may be prepared as described by Mottawia et al, Carbohydr. Res. 277 (1995), 109-123 (which is hereby incorporated by reference) or specifically as described in the “Materials & Methods” section.

A variant of the invention may in an embodiment be capable of hydrolysing starch and other linear and branched 1,4-glucosidic oligo—and polysaccharides and may further have altered (i.e., higher or lower) alpha-1,6-D-glucosidic branch linkage cleavage activity.

In another embodiment the variant of the invention is capable of hydrolysing starch and other linear and branched 1,4-glucosidic oligo—and polysaccharides, but do not have any detectable alpha-1,6-D-glucosidic branch linkage cleavage activity.

### Termamyvl-like Alpha-Amylases

A number of alpha-amylases produced by Bacillus spp. are highly homologous (identical) on amino acid level. The identity of a number of Bacillus alpha-amylases can be found in the below Table 1:

TABLE 1

| | Percent | | | | | | | |
|---|---|---|---|---|---|---|---|---|
| | identity | | | | | | | |
| | 707 | AP1378 | BAN | BSG | SP690 | SP722 | AA560 | Termamyl |
| 707 | 100.0 | 86.4 | 66.9 | 66.5 | 87.6 | 86.2 | 95.5 | 68.1 |
| AP1378 | 86.4 | 100.0 | 67.1 | 68.1 | 95.1 | 86.6 | 86.0 | 69.4 |
| BAN | 66.9 | 67.1 | 100.0 | 65.6 | 67.1 | 68.8 | 66.9 | 80.7 |
| BSG | 66.5 | 68.1 | 65.6 | 100.0 | 67.9 | 67.1 | 66.3 | 65.4 |
| SP690 | 87.6 | 95.1 | 67.1 | 67.9 | 100.0 | 87.2 | 87.0 | 69.2 |
| SP722 | 86.2 | 86.6 | 68.8 | 67.1 | 87.2 | 100.0 | 86.8 | 70.8 |
| AA560 | 95.5 | 86.0 | 66.9 | 66.3 | 87.0 | 86.8 | 100.0 | 68.3 |
| Termamyl | 68.1 | 69.4 | 80.7 | 65.4 | 69.2 | 70.8 | 68.3 | 100.0 |

For instance, the B. licheniformis alpha-amylase comprising the amino acid sequence shown in SEQ ID NO: 8 (commercially available as Termamyl™) has been found to be about 81% homologous (identical) to the B. amyloliquefaciens alpha-amylase comprising the amino acid sequence shown in SEQ ID NO: 10 and about 65% homologous with the B. stearothermophilus alpha-amylase comprising the amino acid sequence shown in SEQ ID NO: 6. Further homologous parent alpha-amylases include SP690 and SP722, respectively, disclosed in WO 95/26397 and further depicted in SEQ ID NO: 2 and SEQ ID NO: 4, respectively, herein. Other Termamyl-like alpha-amylases are the AA560 alpha-amylase derived from Bacillus sp. and shown in SEQ ID NO: 12, and the #707 alpha-amylase derived from Bacillus sp., shown in SEQ ID NO: 13 and described by Tsukamoto et al., Biochemical and Biophysical Research Communications, 151 (1988), pp. 25-31. The Termamyl-like alpha-amylase referred to are KSM AP1378 is disclosed in WO 97/00324 (from KAO Corporation, JP).

Still further homologous Termamyl-like alpha-amylases include the alpha-amylase produced by the B. licheniformis strain described in EP 0252666 (ATCC 27811), and the alpha-amylases identified in WO 91/00353 and WO 94/18314. Other commercially available Termamyl-like alpha-amylases are comprised in the products sold under the following tradenames: Optitherm™ and Takatherm™ (available from Solvay); Maxamyl™ (available from Gist-brocades(DSM)/Genencor), Spezym AA™ and Spezyme Delta AA™ (available from Genencor), and Keistase™ (available from Daiwa), PURASTAR [symbol] ST 5000E, PURASTRAR [symbol] HPAM L (from Genencor Int.).

Because of the substantial homology found between these alpha-amylases, they are considered to belong to the same class of alpha-amylases, namely the above-mentioned class of “Termamyl-like alpha-amylases”.

Accordingly, in the present context, the term “Termamyl-like alpha-amylase” is intended to indicate an alpha-amylase, which, at the amino acid level, exhibits a substantial identity to Termamyl™, i.e., the B. licheniformis apha-amylase having the amino acid sequence shown in SEQ ID NO: 8 herein. In other words, all the following alpha-amylases, which has the amino acid sequences shown in SEQ ID NOS: 2, 4, 6, 8, 10, 12 and 13 herein are considered to be “Termamyl-like alpha-amylase”. Other Termamyl-like alpha-amylases are alpha-amylases i) which displays at least 60%, such as at least 70%, e.g., at least 75%, or at least 80%, at least 85%, at least 90%, at least 95%, at least 97%, at least 98%, at least 99% homology (identity) with at least one of the amino acid sequences shown in SEQ ID NOS: 2, 4, 6, 8, 10, 12, and 13, and/or ii) is encoded by a DNA sequence which hybridizes to the DNA sequences encoding the above-specified alpha-amylases which are apparent from SEQ ID NOS: 1, 3, 5, 7, 9, and of the present specification (which encoding sequences encode the amino acid sequences shown in SEQ ID NOS: 2, 4, 6, 8, 10 and 12 herein, respectively)

In connection with property i), the term “homology” may be determined as the degree of “identity” between the two sequences indicating a derivation of the first sequence from the second. The homology (identity) may suitably be determined by means of computer programs known in the art such as GAP provided in the GCG program package (described above). Thus, Gap GCGv8 may be used with the following default parameters: GAP creation penalty of 5.0 and GAP extension penalty of 0.3, default scoring matrix, for nucleic sequences and 3.0 and 0.1, respectively, from protein sequences. GAP uses the method of Needleman/Wunsch/Sellers to make alignments.

A structural alignment between, e.g., Termamyl® (SEQ ID NO: 8) and a Termamyl-like alpha-amylase may be used to identify equivalent/corresponding positions in other Termamyl-like alpha-amylases. One method of obtaining said structural alignment is to use the Pile Up programme from the GCG package using default values of gap penalties, i.e., a gap creation penalty of 3.0 and gap extension penalty of 0.1. Other structural alignment methods include the hydrophobic cluster analysis (Gaboriaud et al., (1987), FEBS LETTERS 224, pp. 149-155) and reverse threading (Huber, T; Torda, AE, PROTEIN SCIENCE Vol. 7, No. 1 pp. 142-149 (1998).

### Hybridisation

The oligonucleotide probe used in the characterisation of the Termamyl-like alpha-amylase in accordance with property ii) above may suitably be prepared on the basis of the full or partial nucleotide or amino acid sequence of the alpha-amylase in question.

Suitable conditions for testing hybridisation involve pre-soaking in 5xSSC and prehybridizing for 1 hour at ^{˜}40° C. in a solution of 20% formamide, 5xDenhardt's solution, 50 mM sodium phosphate, pH 6.8, and 50 mg of denatured sonicated calf thymus DNA, followed by hybridisation in the same solution supplemented with 100mM ATP for 18 hours at ^{˜}40° C., followed by three times washing of the filter in 2xSSC, 0.2% SDS at 40° C. for 30 minutes (low stringency), preferred at 50° .C (medium stringency), more preferably at 65° C. (high stringency), even more preferably at ^{˜}75° C. (very high stringency). More details about the hybridisation method can be found in Sambrook et al., Molecular Cloning: A Laboratory Manual, 2nd Ed., Cold Spring Harbor, 1989.

In the present context, “derived from” is intended not only to indicate an alpha-amylase produced or producible by a strain of the organism in question, but also an alpha-amylase encoded by a DNA sequence isolated from such strain and produced in a host organism transformed with said DNA sequence. Finally, the term is intended to indicate an alpha-amylase, which is encoded by a DNA sequence of synthetic and/or cDNA origin and which has the identifying characteristics of the alpha-amylase in question. The term is also intended to indicate that the parent alpha-amylase may be a variant of a naturally occurring alpha-amylase, i.e., a variant, which is the result of a modification (insertion, substitution, deletion) of one or more amino acid residues of the naturally occurring alpha-amylase.

### Parent Termamyl-like Alpha-Amylases

According to the invention all Termamy-like alpha-amylases, as defined above, may be used as the parent (i.e., backbone) alpha-amylase. In a preferred embodiment of the invention the parent alpha-amylase is derived from B. lichenifo-rmis, e.g., one of those referred to above, such as the B. licheniformis alpha-amylase having the amino acid sequence shown in SEQ ID NO: 8.

### Parent Hybrid Termamyl-Like Alpha-Amylases

The parent alpha-amylase (i.e., backbone alpha-amylase) may also be a hybrid alpha-amylase, i.e., an alpha-amylase, which comprises a combination of partial amino acid sequences derived from at least two alpha-amylases.

The parent hybrid alpha-amylase may be one, which on the basis of amino acid homology (identity) and/or DNA hybridization (as defined above) can be determined to belong to the Termamyl-like alpha-amylase family. In this case, the hybrid alpha-amylase is typically composed of at least one part of a Termamyl-like alpha-amylase and part(s) of one or more other alpha-amylases selected from Termamyl-like alpha-amylases or non-Termamyl-like alpha-amylases of microbial (bacterial or fungal) and/or mammalian origin.

Thus, the parent hybrid alpha-amylase may comprise a combination of partial amino acid sequences deriving from at least two Termamyl-like alpha-amylases, or from at least one Termamyl-like and at least one non-Termamyl-like bacterial alpha-amylase, or from at least one Termamyl-like and at least one fungal alpha-amylase. The Termamyl-like alpha-amylase from which a partial amino acid sequence derives, may be any of those specific Termamyl-like alpha-amylase referred to herein.

For instance, the parent alpha-amylase may comprise a C-terminal part of an alpha-amylase derived from a strain of B. licheniformis, and a N-terminal part of an alpha-amylase derived from a strain of B. amyloliquefaciens or from a strain of B. stearothermophilus. For instance, the parent alpha-amylase may comprise at least 430 amino acid residues of the C-terminal part of the B. licheniformis alpha-amylase, and may, e.g., comprise a) an amino acid segment corresponding to the 37 N-terminal amino acid residues of the B. amyloliquefaciens alpha-amylase having the amino acid sequence shown in SEQ ID NO: 10 and an amino acid segment corresponding to the 445 C-terminal amino acid residues of the B. licheniformis alpha-amylase having the amino acid sequence shown in SEQ ID NO: 8, or a hybrid Termamyl-like alpha-amylase being identical to the Termamyl sequence, i.e., the Bacillus licheniformis alpha-amylase (BLA) shown in SEQ ID NO: 8, except that the N-terminal 35 amino acid residues (of the mature protein) has been replaced by the N-terminal 33 residues of BAN (mature protein), i.e., the Bacillus amyloliquefaciens alpha-amylase (BAN) shown in SEQ ID NO: 10; or b) an amino acid segment corresponding to the 68 N-terminal amino acid residues of the B. stearothermophilus alpha-amylase (BSG) having the amino acid sequence shown in SEQ ID NO: 6 and an amino acid segment corresponding to the 415 C-terminal amino acid residues of the B. licheniformis alpha-amylase having the amino acid sequence shown in SEQ ID NO: 8.

In a preferred embodiment of the invention the parent Termamyl-like alpha-amylase is a hybrid alpha-amylase of SEQ ID NO: 8 and SEQ ID NO: 10. Specifically, the parent hybrid Termamyl-like alpha-amylase may be a hybrid alpha-amylase comprising the 445 C-terminal amino acid residues of the B. licheniformis alpha-amylase shown in SEQ ID NO: 4 and the 37 N-terminal amino acid residues of the alpha-amylase derived from B. amyloliquefaciens shown in SEQ ID NO: 5, which may suitably further have the following mutations: H156Y+A181T+N190F+A209V+Q264S (using the numbering in SEQ ID NO: 8). The latter mentioned hybrid is referred to as LE174.

Another suitable parent hybrid alpha-amylase is the one previously described in WO 96/23874 (from Novozymes) constituting the N-terminus of BAN, Bacillus amyloliquefaciens alpha-amylase (amino acids 1-300 of the mature protein) and the C-terminus from Termamyl® (amino acids 301-483 of the mature protein). Other specifically contemplated parent alpha-amylase include LE174 with fewer mutations, i.e., the right above mentioned hydrid having the following mutations: A181T+N190F+A209V+Q264S; N190F+A209V+Q264S; A209V+Q264S; Q264S; H156Y+N190F+A209V+Q264S; H156Y+A209V+Q264S; H156Y+Q264S; H156Y+A181T+A209V+Q264S; H156Y+A181T+Q264S; H156Y+Q264S; H156Y+A181T+N190F+Q264S; H156Y+A181T+N190F; H156Y+A181T+N190F+A209V. These hybrids are also considered to be part of the invention.

In a preferred embodiment the parent Termamyl-like alpha amylase is LE174, SP722, or AA560 including any of LE174+G48A+T49I+G107A+I201F; LE174+M197L; LE174+G48A+T49I+G107A+M197L+I201F, or SP722+D183*+G184*; SP722+D183*+G184*+N195F; SP722+D183*+G184*+M202L; SP722+D183*+G184*+N195F+M202L; BSG+I181*+G182*; BSG+I181*+G182*+N193F; BSG+Il81*+G182*+M20OL; BSG+I181*+G182*+N193F+M200L; AA560+D183*+G184*; AA560+D183*+G184*+N195F; AA560+D183*+G184*+M202L; AA560+D183*+G184*+N195F+M202L.

### General mutations in variants of the invention

The particularly interesting amino acid substitutions are those that increase the mobility around the active site of the enzyme. This is accomplished by changes that disrupt stabilizing interaction in the vicinity of the active site, i.e., within preferably 10Å or 8Å or 6Å or 4Å from any of the residues constituting the active site.

Examples are mutations that reduce the size of side chains, such as

Ala to Gly,

s Val to Ala or Gly,

Ile or Leu to Val, Ala, or Gly

Thr to Ser

Such mutations are expected to cause increased flexibility in the active site region either by the introduction of cavities or by the structural rearrangements that fill the space left by the mutation.

It may be preferred that a variant of the invention comprises one or more modifications in addition to those outlined above. Thus, it may be advantageous that one or more Proline residues present in the part of the alpha-amylase variant which is modified is/are replaced with a non-Proline residue which may be any of the possible, naturally occurring non-Proline residues, and which preferably is an Alanine, Glycine, Serine, Threonine, Valine or Leucine.

Analogously, it may be preferred that one or more Cysteine residues present among the amino acid residues with which the parent alpha-amylase is modified is/are replaced with a non-Cysteine residue such as Serine, Alanine, Threonine, Glycine, Valine or Leucine.

Furthermore, a variant of the invention may—either as the only modification or in combination with any of the above outlined modifications—be modified so that one or more Asp and/or Glu present in an amino acid fragment corresponding to the amino acid fragment 185-209 of SEQ ID NO: 10 is replaced by an Asn and/or Gln, respectively. Also of interest is the replacement, in the Termamyl-like alpha-amylase, of one or more of the Lys residues present in an amino acid fragment corresponding to the amino acid fragment 185-209 of SEQ ID NO: 10 by an Arg.

It will be understood that the present invention encompasses variants incorporating two or more of the above outlined modifications.

Furthermore, it may be advantageous to introduce further mutations the variant of the invention. Contemplated are one or more of the following positions (using SEQ ID NO: 8 (Termamyl®) for the numbering):

M15, V128, A111, H133, W138, T149, M197, N188, A209, A210, H405, T412, in particular the following single, double or triple or multi mutations:

M15X, in particular M15T,L;

V128X, in particular V128E;

H133X, in particular H133Y;

N188X, in particular N188S,T,P;

M197X, in particular M197T,L;

A209X, in particular A209V;

M197T/W138F; M197T/W138Y; M15T/H133Y/N188S;

M15/V128E/H133Y/N188S; E119C/S130C; D124C/R127C; H133Y/T149I;

G475R, H133Y/S187D; H133Y/A209V.

### Methods for Preparing Alpha-Amylase Variants of the Invention

Several methods for introducing mutations into genes are known in the art. After a brief discussion of the cloning of alpha-amylase-encoding DNA sequences, methods for generating mutations at specific sites within the alpha-amylase-encoding sequence will be discussed.

### Cloning a DNA Sequence Encoding an Alpha-Amylase

The DNA sequence encoding a parent alpha-amylase may be isolated from any cell or microorganism producing the alpha-amylase in question, using various methods well known in the art. First, a genomic DNA and/or cDNA library should be constructed using chromosomal DNA or messenger RNA from the organism that produces the alpha-amylase to be studied. Then, if the amino acid sequence of the alpha-amylase is known, homologous, labeled oli-gonucleotide probes may be synthesized and used to identify alpha-amylase-encoding clones from a genomic library prepared from the organism in question. Alternatively, a labeled oligonucleotide probe containing sequences homologous to a known alpha-amylase gene could be used as a probe to identify alpha-amylase-encoding clones, using hybridization and washing conditions of lower stringency.

Yet another method for identifying _{α}-amylase-encoding clones would involve inserting fragments of genomic DNA into an expression vector, such as a plasmid, transforming _{α}-amylase-negative bacteria with the resulting genomic DNA library, and then plating the transformed bacteria onto agar containing a substrate for alpha-amylase, thereby allowing clones expressing the alpha-amylase to be identified.

Alternatively, the DNA sequence encoding the enzyme may be prepared synthetically by established standard methods, e.g., the phosphoroamidite method described by S. L. Beaucage and M. H. Caruthers, Tetrahedron Letters 22, 1981, pp. 1859-1869, or the method described by Matthes et al., The EMBO J. 3, 1984, pp. 801-805. In the phosphoroamidite method, oligonucleotides are synthesized, e.g., in an automatic DNA synthesizer, purified, annealed, ligated and cloned in appropriate vectors.

Finally, the DNA sequence may be of mixed genomic and synthetic origin, mixed synthetic and cDNA origin or mixed genomic and cDNA origin, prepared by ligating fragments of synthetic, genomic or cDNA origin (as appropriate, the fragments corresponding to various parts of the entire DNA sequence), in accordance with standard techniques. The DNA sequence may also be prepared by polymerase chain reaction (PCR) using specific primers, for instance as described in US 4,683,202 or R.K. Saiki et al., Science 239, 1988, pp. 487-491.

### Site-Directed Mutagenesis

Once an alpha-amylase-encoding DNA sequence has been isolated, and desirable sites for mutation identified, mutations may be introduced using synthetic oligonucleotides. These oligonucleotides contain nucleotide sequences flanking the desired mutation sites; mutant nucleotides are inserted during oligonucleotide synthesis. In a specific method, a single-stranded gap of DNA, bridging the alpha-amylase-encoding sequence, is created in a vector carrying the alpha-amylase gene. Then the synthetic nucleotide, bearing the desired mutation, is annealed to a homologous portion of the single-stranded DNA. The remaining gap is then filled in with DNA polymerase I (Klenow fragment) and the construct is ligated using T4 ligase. A specific example of this is method is described in Morinaga et al. (1984). US 4,760,025 disclose the introduction of oligonucleotides encoding multiple mutations by performing minor alterations of the cassette. However, an even greater variety of mutations can be introduced at any one time by the Morinaga method, because a multitude of oligonucleotides, of various lengths, can be introduced.

Another method for introducing mutations into alpha-amylase-encoding DNA sequences is described in Nelson and Long (1989). It involves the 3-step generation of a PCR fragment containing the desired mutation introduced by using a chemically synthesized DNA strand as one of the primers in the PCR reactions. From the PCR-generated fragment, a DNA fragment carrying the mutation may be isolated by cleavage with restriction endonucleases and reinserted into an expression plasmid.

### Random Mutagenesis

Random mutagenesis is suitably performed either as localised or region-specific random mutagenesis in at least three parts of the gene translating to the amino acid sequence shown in question, or within the whole gene.

The random mutagenesis of a DNA sequence encoding a parent _{α}-amylase may be conveniently performed by use of any method known in the art.

In relation to the above, a further aspect of the present invention relates to a method for generating a variant of a parent alpha-amylase, e.g., wherein the variant exhibits altered or increased thermal stability relative to the parent, the method comprising:

(a) subjecting a DNA sequence encoding the parent (_{α}-amylase to random mutagenesis,

(b) expressing the mutated DNA sequence obtained in step (a) in a host cell, and

(c) screening for host cells expressing an _{α}-amylase variant which has an altered property (i.e. thermal stability) relative to the parent _{α}-amylase.

Step (a) of the above method of the invention is preferably performed using doped primers.

For instance, the random mutagenesis may be performed by use of a suitable physical or chemical mutagenizing agent, by use of a suitable oligonucleotide, or by subjecting the DNA sequence to PCR generated mutagenesis. Furthermore, the random mutagenesis may be performed by use of any combination of these mutagenizing agents. The mutagenizing agent may, e.g., be one which induces transitions, transversions, inversions, scrambling, deletions, and/or insertions.

Examples of a physical or chemical mutagenizing agent suitable for the present purpose include ultraviolet (UV) ir-radiation, hydroxylamine, N-methyl-N′-nitro-N-nitrosoguanidine (MNNG), O-methyl hydroxylamine, nitrous acid, ethyl methane sulphonate (EMS), sodium bisulphite, formic acid, and nucleotide analogues. When such agents are used, the mutagenesis is typically performed by incubating the DNA sequence encoding the parent enzyme to be mutagenized in the presence of the mutagenizing agent of choice under suitable conditions for the mutagenesis to take place, and selecting for mutated DNA having the desired properties.

When the mutagenesis is performed by the use of an oligonucleotide, the oligonucleotide may be doped or spiked with the three non-parent nucleotides during the synthesis of the oligonucleotide at the positions, which are to be changed. The doping or spiking may be done so that codons for unwanted amino acids are avoided. The doped or spiked oligonucleotide can be incorporated into the DNA encoding the alpha-amylase enzyme by any published technique, using, e.g., PCR, LCR or any DNA polymerase and ligase as deemed appropriate.

Preferably, the doping is carried out using “constant random doping”, in which the percentage of wild-type and mutation in each position is predefined. Furthermore, the doping may be directed toward a preference for the introduction of certain nucleotides, and thereby a preference for the introduction of one or more specific amino acid residues. The doping may be made, e.g., so as to allow for the introduction of 90% wild type and 10% mutations in each position. An additional consideration in the choice of a doping scheme is based on genetic as well as protein-structural constraints. The doping scheme may be made by using the DOPE program, which, inter alia, ensures that introduction of stop codons is avoided.

When PCR-generated mutagenesis is used, either a chemically treated or non-treated gene encoding a parent alpha-amylase is subjected to PCR under conditions that increase the mis-incorporation of nucleotides (Deshler 1992; Leung et al., Technique, Vol.1, 1989, pp. 11-15).

A mutator strain of E. coli (Fowler et al., Molec. Gen. Genet., 133, 1974, pp. 179-191), S. cereviseae or any other microbial organism may be used for the random mutagenesis of the DNA encoding the alpha-amylase by, e.g., transforming a plasmid containing the parent glycosylase into the mutator strain, growing the mutator strain with the plasmid and isolating the mutated plasmid from the mutator strain. The mutated plasmid may be subsequently transformed into the expression organism.

The DNA sequence to be mutagenized may be conveniently present in a genomic or cDNA library prepared from an organism expressing the parent alpha-amylase. Alternatively, the DNA sequence may be present on a suitable vector such as a plasmid or a bacteriophage, which as such may be incubated with or otherwise exposed to the mutagenising agent. The DNA to be mutagenized may also be present in a host cell either by being integrated in the genome of said cell or by being present on a vector harboured in the cell. Finally, the DNA to be mutagenized may be in isolated form. It will be understood that the DNA sequence to be subjected to random mutagenesis is preferably a cDNA or a genomic DNA sequence.

In some cases it may be convenient to amplify the mutated DNA sequence prior to performing the expression step b) or the screening step c). Such amplification may be performed in accordance with methods known in the art, the presently preferred method being PCR-generated amplification using oligonucleotide primers prepared on the basis of the DNA or amino acid sequence of the parent enzyme.

Subsequent to the incubation with or exposure to the mutagenising agent, the mutated DNA is expressed by culturing a suitable host cell carrying the DNA sequence under conditions allowing expression to take place. The host cell used for this purpose may be one which has been transformed with the mutated DNA sequence, optionally present on a vector, or one which was carried the DNA sequence encoding the parent enzyme during the mutagenesis treatment. Examples of suitable host cells are the following: gram positive bacteria such as Bacillus subtilis, Bacillus licheniformis, Bacillus lentus, Bacillus brevis, Bacillus stearothermcphilus, Bacillus alkalophilus, Bacillus amryloliquefaciens, Bacillus coagulans, Bacillus circulans, Bacillus lautus, Bacillus megaterium, Bacillus thuringiensis, Streptomyces lividans or Streptomyces murinus; and gram-negative bacteria such as E. coli.

The mutated DNA sequence may further comprise a DNA sequence encoding functions permitting expression of the mutated DNA sequence.

### Localized Random Mutagenesis

The random mutagenesis may be advantageously localized to a part of the parent alpha-amylase in question. This may, e.g., be advantageous when certain regions of the enzyme have been identified to be of particular importance for a given property of the enzyme, and when modified are expected to result in a variant having improved properties. Such regions may normally be identified when the tertiary structure of the parent enzyme has been elucidated and related to the function of the enzyme.

The localized, or region-specific, random mutagenesis is conveniently performed by use of PCR generated mutagenesis techniques as described above or any other suitable technique known in the art. Alternatively, the DNA sequence encoding the part of the DNA sequence to be modified may be isolated, e.g., by insertion into a suitable vector, and said part may be subsequently subjected to mutagenesis by use of any of the mutagenesis methods discussed above.

### Alternative Methods of Providing Alpha-Amylase Variants

Alternative methods for providing variants of the invention include gene-shuffling method known in the art including the methods, e.g., described in WO 95/22625 (from Affymax Technologies N.V.) and WO 96/00343 (from Novozymes A/S).

### Expression of Alpha-Amylase Variants

According to the invention, a DNA sequence encoding the variant produced by methods described above, or by any alternative methods known in the art, can be expressed, in enzyme form, using an expression vector which typically includes control sequences encoding a promoter, operator, ribosome binding site, translation initiation signal, and, optionally, a repressor gene or various activator genes.

The recombinant expression vector carrying the DNA sequence encoding an alpha-amylase variant of the invention may be any vector, which may conveniently be subjected to recombinant DNA procedures, and the choice of vector will often depend on the host cell into which it is to be introduced. Thus, the vector may be an autonomously replicating vector, i.e., a vector which exists as an extrachromosomal entity, the replication of which is independent of chromosomal replication, e.g., a plasmid, a bacteriophage or an extrachromosomal element, minichromosome or an artificial chromosome. Alternatively, the vector may be one which, when introduced into a host cell, is integrated into the host cell genome and replicated together with the chromosome(s) into which it has been integrated.

In the vector, the DNA sequence should be operably connected to a suitable promoter sequence. The promoter may be any DNA sequence, which shows transcriptional activity in the host cell of choice and may be derived from genes encoding proteins either homologous or heterologous to the host cell. Examples of suitable promoters for directing the transcription of the DNA sequence encoding an alpha-amylase variant of the invention, especially in a bacterial host, are the promoter of the lac operon of E.coli, the Streptomyces coelicolor agarase gene dagA promoters, the promoters of the Bacillus licheniformis alpha-amylase gene (amyL), the promoters of the Bacillus stearothermophilus maltogenic amylase gene (amyM), the promoters of the Bacillus amylolique-faciens alpha-amylase (amyQ), the promoters of the Bacillus subtilis xylA and xylB genes etc. For transcription in a fungal host, examples of useful promoters are those derived from the gene encoding A. oryzae TAKA amylase, Rhizomucor miehei aspartic proteinase, A. niger neutral alpha-amylase, A. niger acid stable alpha-amylase, A. niger glucoamylase, Rhizomucor miehei lipase, A. oryzae alkaline protease, A. oryzae triose phosphate isomerase or A. nidulans acetamidase.

The expression vector of the invention may also comprise a suitable transcription terminator and, in eukaryotes, polyadenylation sequences operably connected to the DNA sequence encoding the alpha-amylase variant of the invention. Termination and polyadenylation sequences may suitably be derived from the same sources as the promoter.

The vector may further comprise a DNA sequence enabling the vector to replicate in the host cell in question. Examples of such sequences are the origins of replication of plasmids pUC19, pACYC177, pUB110, pE194, pAMB1 and pIJ702.

The vector may also comprise a selectable marker, e.g. a gene the product of which complements a defect in the host cell, such as the dal genes from B. subtilis or B. licheniformis, or one which confers antibiotic resistance such as ampicillin, kanamycin, chloramphenicol or tetracyclin resistance. Furthermore, the vector may comprise Aspergillus selection markers such as amdS, argE, niaD and sC, a marker giving rise to hygromycin resistance, or the selection may be accomplished by co-transformation, e.g., as described in WO 91/17243.

While intracellular expression may be advantageous in some respects, e.g., when using certain bacteria as host cells, it is generally preferred that the expression is extracellular. In general, the Bacillus _{α}-amylases mentioned herein comprise a pre-region permitting secretion of the expressed protease into the culture medium. If desirable, this preregion may be replaced by a different preregion or signal sequence, conveniently accomplished by substitution of the DNA sequences encoding the respective preregions.

The procedures used to ligate the DNA construct of the invention encoding an alpha-amylase variant, the promoter, terminator and other elements, respectively, and to insert them into suitable vectors containing the information necessary for replication, are well known to persons skilled in the art (cf., for instance, Sambrook et al., Molecular Cloning: A Laboratory Manual, 2nd Ed., Cold Spring Harbor, 1989).

The cell of the invention, either comprising a DNA construct or an expression vector of the invention as defined above, is advantageously used as a host cell in the recombinant production of an alpha-amylase variant of the invention. The cell may be transformed with the DNA construct of the invention encoding the variant, conveniently by integrating the DNA construct (in one or more copies) in the host chromosome. This integration is generally considered to be an advantage as the DNA sequence is more likely to be stably maintained in the cell. Integration of the DNA constructs into the host chromosome may be performed according to conventional methods, e.g., by homologous or heterologous recombination. Alternatively, the cell may be transformed with an expression vector as described above in connection with the different types of host cells.

The host cell of the invention may be a cell of a higher organism such as a mammal or an insect, but is preferably a microbial cell, e.g., a bacterial or a fungal (including yeast) cell.

Examples of suitable bacteria are Gram-positive bacteria such as Bacillus subtilis, Bacillus licheniformis, Bacillus lentus, Bacillus brevis, Bacillus stearothermophilus, Bacillus alkalo-philus, Bacillus amyloliquefaciens, Bacillus coagulans, Bacillus circulans, Bacillus lautus, Bacillus megaterium, Bacillus thuringiensis, or Streptomyces lividans or Streptomyces murinus, or gramnegative bacteria such as E.coli. The transformation of the bacteria may, for instance, be effected by protoplast transformation or by using competent cells in a manner known per se.

The yeast organism may favorably be selected from a species of Saccharomyces or Schizosaccharomyces, e.g. Saccharomyces cerevisiae. The filamentous fungus may advantageously belong to a species of Aspergillus, e.g., Aspergillus oryzae or Aspergillus niger. Fungal cells may be transformed by a process involving protoplast formation and transformation of the protoplasts followed by regeneration of the cell wall in a manner known per se. A suitable procedure for transformation of Aspergillus host cells is described in EP 238 023.

In a yet further aspect, the present invention relates to a method of producing an alpha-amylase variant of the invention, which method comprises cultivating a host cell as described above under conditions conducive to the production of the variant and recovering the variant from the cells and/or culture medium.

The medium used to cultivate the cells may be any conventional medium suitable for growing the host cell in question and obtaining expression of the alpha-amylase variant of the invention. Suitable media are available from commercial suppliers or may be prepared according to published recipes (e.g., as described in catalogues of the American Type Culture Collection).

The alpha-amylase variant secreted from the host cells may conveniently be recovered from the culture medium by well-known procedures, including separating the cells from the medium by centrifugation or filtration, and precipitating proteinaceous components of the medium by means of a salt such as ammonium sulphate, followed by the use of chromatographic procedures such as ion exchange chromatography, affinity chromatography, or the like.

### Industrial Applications

The alpha-amylase variants of this invention possess valuable properties allowing for a variety of industrial applications. In particular, enzyme variants of the invention are applicable as a component in washing, dishwashing and hard surface cleaning detergent compositions. Variant of the invention with altered properties may be used for starch processes, in particular starch conversion, especially liquefaction of starch (see, e.g., US 25 3,912,590, EP patent publications Nos. 252 730 and 63 909, and WO 99/19467.

Further, variants of the invention are also particularly useful in the production of sweeteners and ethanol from starch, and/or for textile desizing.

### Detergent Compositions

As mentioned above, variants of the invention may suitably be incorporated in detergent compositions. Reference is made, for example, to WO 96/23874 and WO 97/07202 for further details concerning relevant ingredients of detergent compositions (such as laundry or dishwashing detergents), appropriate methods of formulating the variants in such detergent compositions, and for examples of relevant types of detergent compositions.

Detergent compositions comprising a variant of the invention may additionally comprise one or more other enzymes, such as a lipase, cutinase, protease, cellulase, peroxidase or laccase, and/or another alpha-amylase.

Alpha-amylase variants of the invention may be incorporated in detergents at conventionally employed concentrations. It is at present contemplated that a variant of the invention may be incorporated in an amount corresponding to 0.00001-10 mg (calculated as pure, active enzyme protein) of alpha-amylase per liter of wash/dishwash liquor using conventional dosing levels of detergent.

### Materials & Methods

### Enzymes

TERMAMYL®: B. licheniformis alpha-amylase shown in SEQ ID NO: 8. NOVAMYL®: B. stearotherrmophilus C599 maltogenic amylase disclosed in EP patent no. 120,693 (available from Novo Nordisk)

Bacillus subtilis SHA273: see WO 95/10603

### Plasmids

PJE1 contains the gene encoding a variant of SP722 alpha-amylase (SEQ ID NO: 4): viz. deletion of 6 nucleotides corresponding to amino acids D183-G184 in the mature protein. Transcription of the JEl gene is directed from the amyL promoter. The plasmid further more contains the origin of replication and cat-gene conferring resistance towards kanamycin obtained from plasmid pUB110 (Gryczan, TJ et al. (1978), J. Bact. 134:318-329).

### Methods

### Construction of Library Vector PDorK101

The E. colil/Bacillus shuttle vector pDorK101 (described below) can be used to introduce mutations without expression of alpha-amylase in E. coli and then be modified in such way that the alpha-amylase is active in Bacillus. The vector was constructed as follows: The JE1 encoding gene (SP722 with the deletion of D183-G184) was inactivated in pJE1 by gene interruption in the PstI site in the 5′ coding region of the SEQ ID NO: 4: SP722 by a 1.2 kb fragment containing an E. coli origin of replication. This fragment was PCR amplified from the pUC19 (GenBank Accession #:X02514) using the forward primer: 5′-gacctgcagtcaggcaacta-3′ (SEQ ID NO: 14) and the reverse primer: 5′-tagagtcgacctgcaggcat-3′ (SEQ ID NO: 15). The PCR amplicon and the pJE1 vector were digested with PstI at 37° C. for 2 hours. The pJE1 vector fragment and the PCR fragment were ligated at room temperature. for 1 hour and transformed in E. coli by electrotransformation. The resulting vector is designated pDorK101.

### Fermentation and Purification of Alpha-Amylase Variants

Fermentation and purification may be performed by methods well known in the art.

### Procedure for Determination of 1,6-Activity

The enzyme solutions (of a chosen activity, e.g., 10-100 NU) are diluted with D₂O and freeze-dried. The samples are re-dissolved in D₂O (0.5 mL) and freeze-dried. Samples containing 25 mg of substrate in D₂O (0.5 mL) are freeze-dried before re-dissolved (D₂O, 0.5 mL) and freeze-dried. Finally the enzymes are dissolved in D₂O (1 mL) and added to each sample of substrate. The solutions are transferred to NMR tubes and incubated at 60° C. ¹H NMR spectra are recorded currently at 60° C. on a Varian Mercury 400 MHz instrument (5 mm inverse probe head, 32 scan).

### Preparation of NOVAMYL® and TERMAMYL® Limit Dextrins

The NOVAMYL® limit dextrin is prepared from waxy maize starch (100% amylopectin) by gelatinization at 80° C. and treatment with NOVAMYL® for 4 hours at 60° C. The enzyme is inactivated at 100° C. (10 minutes) before smaller dextrins are removed by ultrafiltration. The dextrin is then be obtained as white fibers after freeze-drying.

The Termamyl limit dextrin is prepared by a similar procedure; gelatinisation at 100° C., incubation for 2 hours at 90° C., inactivation, ultrafiltration and freeze-drying. See also Mottawia et al, Carbohydr. Res. 277 (1995), 109-123 (purification and beta-amylase treatment excluded in the present procedure).

### Assays for Alpha-Amylase Activity

1. Phadebas Assay

Alpha-amylase activity is determined by a method employing PHADEBAS® tablets as substrate. Phadebas tablets (PHADEBAS® Amylase Test, supplied by Pharmacia Diagnostic) contain a cross-linked insoluble blue-colored starch polymer, which has been mixed with bovine serum albumin and a buffer substance and tabletted.

For every single measurement one tablet is suspended in a tube containing 5 ml 50 mM Britton-Robinson buffer (50 mM acetic acid, 50 mM phosphoric acid, 50 mM boric acid, 0.1 mM CaCl₂, pH adjusted to the value of interest with NaOH). The test is performed in a water bath at the temperature of interest. The alpha-amylase to be tested is diluted in ×ml of 50 mM Britton-Robinson buffer. 1 ml of this alpha-amylase solution is added to the 5 ml 50 mM Britton-Robinson buffer. The starch is hydrolyzed by the alpha-amylase giving soluble blue fragments. The absorbance of the resulting blue solution, measured spectrophotometrically at 620 nm, is a function of the alpha-amylase activity.

It is important that the measured 620 nm absorbance after 10 or 15 minutes of incubation (testing time) is in the range of 0.2 to 2.0 absorbance units at 620 nm. In this absorbance range there is linearity between activity and absorbance (Lambert-Beer law) The dilution of the enzyme must therefore be adjusted to fit this criterion. Under a specified set of conditions (temp., pH, reaction time, buffer conditions) 1 mg of a given alpha-amylase will hydrolyze a certain amount of substrate and a blue colour will be produced. The colour intensity is measured at 620 nm. The measured absorbance is directly proportional to the specific activity (activity/mg of pure alpha-amylase protein) of the alpha-amylase in question under the given set of conditions.

2. Alternative Method

Alpha-amylase activity is determined by a method employing the PNP-G₇ substrate. PNP-G₇ which is a abbreviation for p-nitrophenyl-alpha,D-maltoheptaoside is a blocked oligosaccharide which can be cleaved by an endo-amylase. Following the cleavage, the alpha-Glucosidase included in the kit digest the substrate to liberate a free PNP molecule which has a yellow colour and thus can be measured by visible spectophometry at _{γ}=405 nm. (400-420 nm.). Kits containing PNP-G₇ substrate and alpha-Glucosidase is manufactured by Boehringer-Mannheim (cat. No. 1054635).

To prepare the substrate one bottle of substrate (BM 1442309) is added to 5 ml buffer (BM1442309). To prepare the _{α}-Glucosidase one bottle of alpha-Glucosidase (BM 1462309) is added to 45 ml buffer (BM1442309). The working solution is made by mixing 5 ml alpha-Glucosidase solution with 0.5 ml substrate.

The assay is performed by transforming 20 micro 1 enzyme solution to a 96 well microtitre plate and incubating at 25° C. 200 micro 1 working solution, 25° C. is added. The solution is mixed and pre-incubated 1 minute and absorption is measured every 15 second over 3 minutes at OD 405 nm.

The slope of the time dependent absorption-curve is directly proportional to the specific activity (activity per mg enzyme) of the alpha-amylase in question under the given set of conditions.

### General Method for Random Mutagenesis by use of the DOPE Program

The random mutagenesis may be carried out as follows:

1. Select regions of interest for modification in the parent enzyme

2. Decide on mutation sites and non-mutated sites in the selected region

3. Decide on which kind of mutations should be carried out, e.g. with respect to the desired stability and/or performance of the variant to be constructed

4. Select structurally reasonable mutations.

5. Adjust the residues selected by step 3 with regard to step 4.

6. Analyze by use of a suitable dope algorithm the nucleotide distribution.

7. If necessary, adjust the wanted residues to genetic code realism (e.g., taking into account constraints resulting from the genetic code (e.g. in order to avoid introduction of stop codons))(the skilled person will be aware that some codon combinations cannot be used in practice and will need to be adapted)

8. Make primers

9. Perform random mutagenesis by use of the primers

10. Select resulting alpha-amylase variants by screening for the desired improved properties.

Suitable dope algorithms for use in step 6 are well known in the art. One algorithm is described by Tomandl, D. et al., Journal of Computer-Aided Molecular Design, 11 (1997), pp. 29-38). Another algorithm, DOPE, is described in the following:

### The Dope Program

The “DOPE” program is a computer algorithm useful to optimize the nucleotide composition of a codon triplet in such a way that it encodes an amino acid distribution, which resembles most the wanted amino acid distribution. In order to assess which of the possible distributions is the most similar to the wanted amino acid distribution, a scoring function is needed. In the “Dope” program the following function was found to be suited:

$$s ≡ {\prod }_{i = 1}^{N} {( \frac{{x}_{i}^{{y}_{i}}}{{y}_{i}^{{y}_{i}}} \frac{{( 1 - {x}_{i} )}^{1 - {y}_{i}}}{{( 1 - {y}_{i} )}^{1 - {y}_{i}}} )}^{{w}_{i}} ,$$

where the x₁'s are the obtained amounts of amino acids and groups of amino acids as calculated by the program, y₁'s are the wanted amounts of amino acids and groups of amino acids as defined by the user of the program (e.g. specify which of the 20 amino acids or stop codons are wanted to be introduced, e.g., with a certain percentage (e.g. 90% Ala, 3% Ile, 7% Val), and w₁'s are assigned weight factors as defined by the user of the program (e.g., depending on the importance of having a specific amino acid residue inserted into the position in question). N is 21 plus the number of amino acid groups as defined by the user of the program. For purposes of this function 0⁰ is defined as being 1.

A Monte-Carlo algorithm (one example being the one described by Valleau, J. P. & Whittington, S. G. (1977) A guide to Mont Carlo for statistical mechanics: 1 Highways. In “Stastistical Mechanics, Part A” Equlibrium Techniqeues ed. B. J. Berne, New York: Plenum) is used for finding the maximum value of this function. In each iteration the following steps are performed:

1.A new random nucleotide composition is chosen for each base, where the absolute difference between the current and the new composition is smaller than or equal to d for each of the four nucleotides G,A,T,C in all three positions of the codon (see below for definition of d).

2.The scores of the new composition and the current composition are compared by the use of the function s as described above. If the new score is higher or equal to the score of the current composition, the new composition is kept and the current composition is changed to the new one. If the new score is smaller, the probability of keeping the new composition is exp(1000(new_score-current_score)).

A cycle normally consists of 1000 iterations as described above in which d is decreasing linearly from 1 to 0. One hundred or more cycles are performed in an optimization process. The nucleotide composition resulting in the highest score is finally presented.

### EXAMPLES

### Example 1

### Construction of Variants of the Invention and Determination of Altered 1,6-Activity

The following variants are constructed as described in EXAMPLE 1 of WO 00/29560 (from Novozymes A/S) in the Bacillus licheniformis alpha-amylase shown in SEQID NO: 8:

W263GA,S,T,V;

N265G,A,S,T,V;

V286F,W,Y,G,A,S;

Y290A,N,D,C,Q,E,G,H,I,L,K,M,F,P,S,T,V;

L335G,A,S,T,V; and

K234N,Q.

The altered 1,6-activity is determined in comparison to the pared as described in the Materials & Methods″ section above under ″Procedure for determination of 1,6-activity [symbol] .

1. A alpha-amylase variant of a parent Termamyl-like alpha-amylase, comprising an alteration at one or more of the following regions or positions selected from the group consisting of:
186-193,
261-276,
283-293,
334-339,and
234,
wherein each region or position corresponds to a region or position of the amino acid sequence of B. licheniformis alpha-amylase shown in seq id no: 8.

2. The variant of claim 1, wherein the variant has an alteration in one or more of the following positions: W263,E189,L335,Y290,N265,V286,Q264,K234.

3.The variant of claim 1, wherein the variant has the following alterations: W263GASTV; E189GASTV, L335GASTV, Y290 A,N,D,C,Q,E,G,H,I,L,K,M,F,P,S,T,V;N265G,A,S,T,V; V286F,W,Y,G,A,S; Q264X, K234X, preferably NQ.

4. The variant of any of claims 1-3, wherein the parent Termamyl-like alpha-amylase is derived from a strain of B. licheniformis, B. amyloliquefaciens, B. stearothermophilus, Bacillus sp. NCIB 12289, NCIB 12512, NCIB 12513 or DSM 9375, or DSMZ no. 12649, KSM AP1378.

5. The variant of claims 1-4, wherein the parent Termamyl-like alpha-amylase is any of the alpha-amylases selected from the group depicted in SEQ ID NOS: 2, 4, 6, 8, 10, 12, and 13.

6. The variant according to any of claims 1-5, wherein the parent Termamyl-like alpha-amylase has an amino acid sequence which has a degree of identity to SEQ ID NO: 4 of at least 60%.

7. The variant of any of claims 1-6, wherein the parent Termamyl-like alpha-amylase is encoded by a nucleic acid sequence which hydridizes under low stringency conditions with the nucleic acid sequence of SEQ ID NO: 7.

8. The variant of claims 1-7, which variant has altered alpha-1,6-D-glucosidic branch linkage cleavage activity on amylopectin, referably increased alpha-1,6-D-glucosidic branch linkage cleavage activity of amylopectin or a limit dextrin prepared by TERMAMYL™ or NOVAMYL®.

9. A DNA construct comprising a DNA sequence encoding an alpha-amylase variant according to any one of claims 1 to 8.

10. A recombinant expression vector which carries a DNA construct according to claim 9.

11. A cell which is transformed with a DNA construct according to claim 9 or a vector according to claim 10.

12. A cell according to claim 11, which is a microorganism, preferably a bacterium or a fungus.

13. The cell according to claim 12, which cell is a gram positive bacterium, such as Bacillus subtilis, Bacillus licheniformis, Bacillus lentus, Bacillus brevis, Bacillus stearothermophilus, Bacillus alkalophilus, Bacillus amyloliquefaciens, Bacillus coagu-lans, Bacillus circulans, Bacillus lautus or Bacillus thu-ringiensis.

14. A detergent additive comprising an alpha-amylase variant according to any one of claims 1 to 8, optionally in the form of a non-dusting granulate, stabilised liquid or protected enzyme.

15. A detergent additive according to claim 14, which contains 0.02-200 mg of enzyme protein/g of the additive.

16. A detergent additive according to claims 14 or 15, which additionally comprises another enzyme such as a protease, a lipase, a peroxidase, amylase or another amylolytic enzyme, such as glucoamylase, and/or a cellulase.

17. A detergent composition comprising an alpha-amylase variant according to any of claims 1 to 8.

18. A detergent composition according to claim 17, which additionally comprises another enzyme such as a protease, a lipase, a peroxidase, another amylolytic enzyme and/or a cellulase.

19. A manual or automatic dishwashing detergent composition comprising an alpha-amylase variant according to any of claims 1 to 8.

20. A dishwashing detergent composition according to claim 19, which additionally comprises another enzyme such as a protease, a lipase, a peroxidase, amylase or another amylolytic enzyme, such as glucoamylase, and/or a cellulase.

21. A manual or automatic laundry washing composition comprising an alpha-amylase variant according to any of claims 1 to 8.

22. A laundry washing composition according to claim 21, which additionally comprises another enzyme such as a protease, a lipase, a peroxidase, an amylase and/or another an amylolytic enzyme, such as glucoamylase and/or a cellulase.

23. A composition comprising an alpha-amylase variant of any is of claims 1-8.

24. The composition of claims 23, which further comprise another alpha-amylase, glucoamylase, pullulanase, isoamylase, protease, preferably acidic protease, especially from Aspergillus, such as A. niger or A. aculatus.
